(19) United States
(12) Patent Application Publication
(10) Pub. No.: US 2010/0262012 A1
(43) Pub. Date: Oct. 14, 2010

(54) METHOD FOR A MEDICAL DIAGNOSTIC ULTRASOUND SYSTEM TO SAVE POWER AND THE MEDICAL DIAGNOSTIC ULTRASOUND SYSTEM

(76) Inventor: Feng Wu, Wuxi (CN)

Correspondence Address: PATRICK W. RASCHE (20459);ARMSTRONG TEASDALE LLP, 7700 Forsyth Boulevard, Suite 1800, St. Louis, MO, 63105, US

(21) Appl. No.: 12/760,104

(22) Filed: Apr. 14, 2010

(30) Foreign Application Priority Data
Apr. 14, 2009 (CN) 200910132730.6

(51) Int. Cl. A61B 8/00

(52) U.S. Cl. 600/459

(57) ABSTRACT

To save power used by a medical diagnostic ultrasound system, a detection device is disposed on the probe side of the medical diagnostic ultrasound system to detect whether the probe is being used, and to generate a corresponding control signal according to the detected signal. A transmitting and receiving circuit disposed on the probe side transmits the signal to a transmitting and receiving circuit disposed on the host side to control a working mode of an ultrasound transmitting and receiving circuit within the host.

### CROSS REFERENCE TO RELATED APPLICATIONS

This application claims the benefit of Chinese Patent Application No. 200910132730.6 filed Apr. 14, 2009, which is hereby incorporated by reference in its entirety.

### BACKGROUND OF THE INVENTION

The present invention relates to the field of ultrasound medical diagnosis, and in particular to the improvement of small ultrasound devices.

In a portable medical diagnostic ultrasound system, power consumption is the biggest challenge. For a working ultrasound device, about half time is in non-working status, i.e. the probe is left aside, but the transmitting and receiving circuits in the system keep on working. This not only wastes power (especially battery power), but when the probe is not on the patient body, namely when the probe is in non-working status, because the transmitted power is held up on the probe surface due to its inability to dissipate through the body, the temperature of the probe surface will increase very fast, rendering the probe overheat, thus becoming a big problem for designing the probe transmitted pulse amplitude. In general, the system in such a situation must reduce the transmitted voltage to reduce the temperature. However, once the transmitted voltage is reduced, the ultrasound image quality will be reduced accordingly.

### BRIEF DESCRIPTION OF THE INVENTION

According to one aspect of the present invention, a medical diagnostic ultrasound system is provided, which comprises a host and a probe, and further comprises a transmitting and receiving circuit disposed on the host side, a transmitting and receiving circuit disposed on the probe side, a processor disposed on the probe side and a detection device disposed on the probe side, wherein the detection device is for detecting whether the probe is being used; the processor is for monitoring the change of the detection device, and transmitting a control signal to the transmitting and receiving circuit on the probe side according to the detected change, the control signal is transmitted to the transmitting and receiving circuit on the host side, for controlling the working mode of the ultrasound receiving and transmitting circuit within the host.

In the above medical diagnostic ultrasound system, the detection device is an accelerometer or a vibration sensor.

In the above medical diagnostic ultrasound system, the processor can be an ultralow power-consuming processor, especially when the present invention is used in a portable medical diagnostic ultrasound system. The processor can be a separate component, or be disposed within the transmitting and receiving device on the probe side or within the detection device.

In the above medical diagnostic ultrasound system, the transmitting and receiving circuit on the host side is connected via an USB interface to the hose, and it can be disposed either within the host or out of the host on demand

In the above medical diagnostic ultrasound system, the processor in the host receives the control signal from the transmitting and receiving circuit on the host side to control the working mode of the ultrasound receiving and transmitting circuit within the host. Optionally, an ultralow power-consuming processor can be provided on the host side, for receiving the control signal from the transmitting and receiving circuit on the host side, and transmitting to the host to control the working mode of the ultrasound receiving and transmitting circuit within the host. The ultralow power-consuming processor can be a separate component, or be disposed within the transmitting and receiving circuit on the host side or within the host.

In the above medical diagnostic ultrasound system, the transmitting and receiving circuit on the host side can transmit signals with the transmitting and receiving circuit on the probe side in a wired manner or a wireless manner of radio frequency.

In the above medical diagnostic ultrasound system, the transmitting and receiving circuit, ultralow power-consuming processor and accelerometer on the probe side are disposed in one module. Additionally, they can be combined together to form a ring to be sheathed on the probe.

In the above medical diagnostic ultrasound system, the probe can be provided with at least one of the following four function keys: freeze, auto, printing and sensing, wherein the freeze key is for manually controlling the ON and OFF of the ultrasound transmitting and receiving circuit within the host of the medical diagnostic ultrasound system; the auto key is for auto mode, i.e. making the medical diagnostic ultrasound system optimize some settings of the ultrasound image automatically; the printing key is for directly transmitting the ultrasound image to the printer to be printed; the sensing key is for manually controlling whether or not to start the accelerometer to detect the state of the probe; wherein the priority level of the freeze key is higher than the sensing key.

The above medical diagnostic ultrasound system can be a portable medical diagnostic ultrasound system.

According to another aspect of the invention, a method for the medical diagnostic ultrasound system to save power is provided, the method includes providing an accelerometer on the probe side of the medical diagnostic ultrasound system to detect whether the probe is touched or not.

In some embodiments, the method also includes monitoring the change of the accelerometer by an ultralow power-consuming processor on the probe side, and generating a corresponding control signal.

In some embodiments, the method also includes disposing transmitting and receiving circuits respectively on the probe side and the host side of the medical diagnostic ultrasound system, for transmitting the corresponding control signal from the probe side to the host side, to control the ultrasound transmitting and receiving circuits within the host to be in a freeze mode or a scan mode.

According to the above method of the present invention, the transmitting and receiving circuits on the probe side and the host side transmit signals in a wired manner or a wireless manner of radio frequency.

The above method of the present invention is used preferably for a portable medical diagnostic ultrasound system.

The present invention enables a portable ultrasound device to reduce waste of power supply, thereby increasing the working hours of batteries. Additionally, the transmit voltage will not be reduced due to the increase in temperature resulted from the blank scan of the probe, therefore the ultrasound image is improved.

### BRIEF DESCRIPTION OF THE DRAWINGS

FIG. 1 shows a schematic diagram of the medical diagnostic ultrasound system of the present invention;

FIG. 2 shows a schematic diagram of a preferred embodiment of the portable medical diagnostic ultrasound system of the present invention.

### DETAILED DESCRIPTION OF THE INVENTION

The present invention is described in detail with reference to the figures, but the present invention is not limited to the following embodiments.

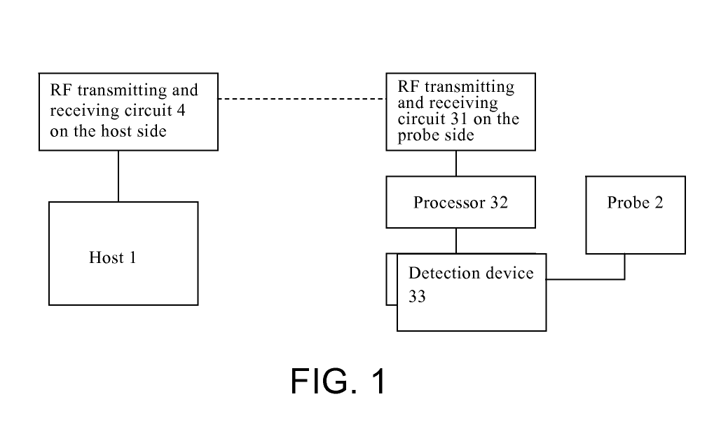

As shown in FIG. 1, RF transmitting and receiving circuits 4 and 31 are disposed respectively on the host 1 side and the probe 2 side of the portable medical diagnostic ultrasound system, and a processor 32 and a detection device 33 are disposed on the probe 2 side, wherein the detection device 33 is for detecting whether the probe is being used; the processor 32 is for monitoring the change of the detection device 33, and transmitting a control signal to the transmitting and receiving circuit 31 on the probe side according to the monitored change, the control signal is transmitted to the transmitting and receiving circuit 4 on the host side. The RF transmitting and receiving circuit 4 on the host side is connected to the host 1 of the portable medical diagnostic ultrasound system, for transmitting the control signal received from the RF transmitting and receiving circuit 31 on the probe side to the host 1 of the portable medical diagnostic ultrasound system, and according to the received control signal, the processor of the host 1 controls the working status of the ultrasound transmitting and receiving circuit in the portable medical diagnostic ultrasound system: freeze status, scan status.

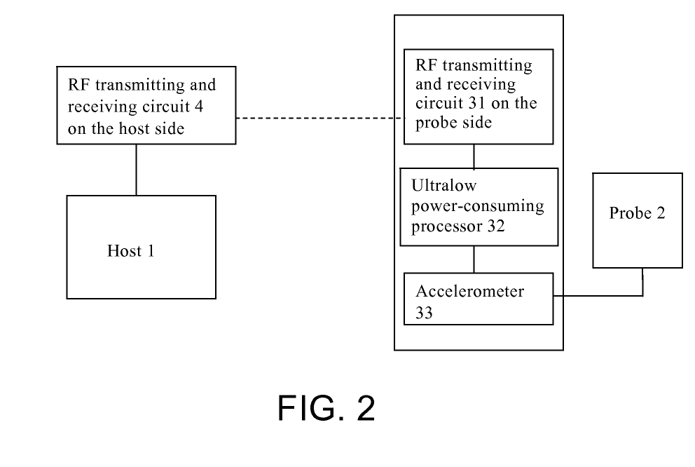

In some embodiments, the detection device 33 is a vibration sensor, preferably an accelerometer and the processor 32 is an ultralow power-consuming processor, especially when the present invention is used for a portable medical diagnostic ultrasound system. FIG. 2 shows a schematic diagram of one embodiment of the portable medical diagnostic ultrasound system of the present invention. The accelerometer 33 is for detecting the status of the probe 2: still or active, namely whether the probe is touched by the doctor or whether the probe is being used. The change of the accelerometer 33 is monitored by the ultralow power-consuming processor 32. If the probe is left aside and is not touched for a predetermined time, the accelerometer 33 detects that the probe is in a still status, the ultralow power-consuming processor 32 then monitors this information of the accelerometer 33 and sends a freeze signal to the RF transmitting and receiving circuit 31 on the probe side. The RF transmitting and receiving circuit 31 on the probe side transmits the freeze signal to the RF transmitting and receiving circuit 4 on the host side, making the ultrasound transmitting and receiving circuit within the host 1 enter a freeze status, namely causing the ultrasound transmitting and receiving circuit within the system to close, thereby saving power consumption. As soon as a doctor picks up the probe or touches the probe, the accelerometer will sense this change, the ultralow power-consuming processor 32 at this time monitors the change of the accelerometer and generates a start signal, which is to be sent wirelessly through radio frequency by the RF transmitting and receiving circuit 31 on the probe side to the RF transmitting and receiving circuit 4 on the host side, then to the host 1 of the medical diagnostic ultrasound system, causing the medical diagnostic ultrasound system to resume working, i.e. making the ultrasound transmitting and receiving circuit enter from a freeze mode into a scan mode. Due to the circuits' fast and automatic response, the whole process is not sensible to the operator.

In designing the above circuits, the ultralow power-consuming processor 32 can be disposed in the accelerometer 33 or in the RF transmitting and receiving circuit 31 on the probe side on demand. Additionally, the accelerometer 33, ultralow power-consuming processor 32, and RF transmitting and receiving circuit 31 on the probe side can be manufactured in one module to form a ring to be sheathed on the probe 2. In addition, the accelerometer 33, ultralow power-consuming processor 32, and RF transmitting and receiving circuit 31 on the probe side or one or two of them can be disposed in the probe 2 as required. The RF signal transmitting and receiving circuit 4 on the host side can be an RF transmitter and receiver with an USB interface inserted in the USB interface on the host side, and is compatible with the current foot switch used in the prior art; it can also be disposed within the host 1 of the portable medical diagnostic ultrasound system. Optionally, the interface here can be an other interface other than the USB interface. In addition, FIG. 1 and FIG. 2 only show the embodiments of transmitting a control signal in the RF wireless transmission manner, but the present invention can also adopt other manners to transmit the control signal, e.g. a wired communication manner. The accelerometer may adopt a semiconductor accelerometer, such as ADI; the RF transmitting and receiving circuits on the host side and the probe side may be, for example RF transmitting and receiving circuits with a frequency of 2.4 G.

In general, the host 1 of the portable medical diagnostic ultrasound system determines via a processor the working status of the ultrasound receiving and transmitting circuit within the host according to the control signal transmitted from the RF transmitting and receiving circuit 4 on the host side: scan mode or freeze mode. Additionally, an ultralow power-consuming processor can be disposed outside the host on the host side to process the control signal transmitted by the RF transmitting and receiving circuit 31 on the probe side via the RF transmitting and receiving circuit 4 on the host side, and control the working status of the ultrasound receiving and transmitting circuit within the host via the processor within the host. Optionally, the ultralow power-consuming processor can be disposed in the RF transmitting and receiving circuit 4 on the host side.

If the portable medical diagnostic ultrasound system 1 enters the freeze mode, namely the ultrasound transmitting and receiving circuit in the system enters the freeze mode, the RF transmitting and receiving circuits 4 and 31 on the host side and the probe side will get into a standby mode to wait for new signals. Because the RF transmitting and receiving circuits themselves do not require much power consumption, and the ultrasound transmitting and receiving circuits in the system are the main power consuming parts of the system, strictly controlling the power consumption of the latter can achieve the effect of saving power, and put the probe surface temperature under proper control, namely the temperature of the probe will not increase too high or too fast due to idling, but can be kept in basic stability, so does not need to reduce the transmitted voltage, thus ensure the quality of the ultrasound image.

Additionally, in order to facilitate operation for the doctor or other users, four function keys can be provided on the medical diagnostic ultrasound system of the present invention: freeze, auto, printing, and sensing, wherein the freeze key is for manually controlling the ON and OFF of the ultrasound transmitting and receiving circuit in the host 1 of the portable medical diagnostic ultrasound system. A press on the key makes the portable medical diagnostic ultrasound system 1 into the freeze mode, another press on the key makes the portable medical diagnostic ultrasound system 1 into the scan mode. The freeze key in the prior art is arranged on the host side of the portable medical diagnostic ultrasound system, so is not handy when a doctor using the probe is far away from the freeze key. The auto key is for auto mode, i.e. making the medical diagnostic ultrasound system optimize some settings of the ultrasound image automatically, e.g. gains, focal positions and so on. The printing key is for directly transmitting the ultrasound image to the printer to be printed. The sensing key is for manually controlling whether or not to start the auto detection function, namely whether or not to enable the accelerometer 33 to detect the state of the probe. Additionally, the priority level of the freeze key is higher than the sensing key, e.g. when the sensing key is pressed to enable the accelerometer, the operator can press the freeze key to manually send the system into the freeze mode. The control signals generated by these function keys can be transmitted via the RF transmitting and receiving circuits 31 and 4 on the probe side and the host side in a wired manner or a wireless manner of radio frequency to the host side to achieve respective functions.

The present invention achieves transmission of control signals by the use of an accelerometer and in a wireless manner of radio frequency, detects the working status of the portable medical diagnostic ultrasound system with fast and automatic response, making it enter the freeze mode or scan mode automatically, thus reducing the waste of power supply when the portable ultrasound device is not in the scan phase, and improving the battery working hours, so that the probe surface temperature will not overheat due to departure from the object under detection, then the transmitted voltage is put under strict control, and the image quality is improved thereby.

The present invention can be applied not only to a medical diagnostic ultrasound system, but also to any ultrasound devices that need to save power.

Some of the above functions of the present invention can be implemented in software or hardware or a combination of software and hardware. In the above description, the “a” used before certain component does not exclude the use of many such components.

The above description is the embodiments of the present invention. It should be noted that, a person having ordinary skill in the art, without having to depart from the spirit of the present invention, can make certain improvement, amendment, and variation, so all these improvement, amendment, and variation shall be deemed as falling within the scope of protection of the present application.

1. A medical diagnostic ultrasound system comprising:
a host comprising an ultrasound transmitting and receiving circuit;
a probe communicatively coupled to the host;
a first transmitting and receiving circuit disposed on a host, side of the medical diagnostic ultrasound system; and
a second transmitting and receiving circuit, a processor, and a detection device disposed on the probe side of the medical diagnostic ultrasound system, wherein:
the detection device is configured to detect whether the probe is being used;
the processor is configured to monitor a change of the detection device and to transmit a control signal to the second transmitting and receiving circuit according to the monitored change, the control signal being transmitted to the first transmitting and receiving circuit for controlling a working mode of the ultrasound receiving and transmitting circuit.

2. The medical diagnostic ultrasound system according to claim 1, wherein the detection device comprises one of an accelerometer and a vibration sensor.

3. The medical diagnostic ultrasound system according to claim 1, wherein the processor comprises an ultralow power-consuming processor.

4. The medical diagnostic ultrasound system according to claim 1, wherein the processor is disposed within the second transmitting and receiving circuit.

5. The medical diagnostic ultrasound system according to claim 1, wherein the processor is disposed within the detection device.

6. The medical diagnostic ultrasound system according to claim 1, wherein the second transmitting and receiving circuit, the processor, and the detection device are disposed in one module.

7. The medical diagnostic ultrasound system according to claim 1, wherein the second transmitting and receiving circuit, the processor, and the detection device are combined together to form a ring so as to be sheathed on the probe.

8. The medical diagnostic ultrasound system according to claim 1, wherein the first transmitting and receiving circuit is connected to the host via an USB interface.

9. The medical diagnostic ultrasound system according to claim 1, wherein the first transmitting and receiving circuit is disposed within the host.

10. The medical diagnostic ultrasound system according to claim 1, wherein the host comprises a processor configured to receive the control signal from the first transmitting and receiving circuit and to control the working mode of the ultrasound receiving and transmitting circuit.

11. The medical diagnostic ultrasound system according to claim 1, further comprising an ultralow power-consuming processor disposed on the host side, the ultralow power-consuming processor configured to receive the control signal from the first transmitting and receiving circuit and to control the working mode of the ultrasound receiving and transmitting circuit.

12. The medical diagnostic ultrasound system according to claim 1, wherein the first transmitting and receiving circuit comprises an ultralow power-consuming processor configured to process the control signal received by the first transmitting and receiving circuit and to transmit the processed control signal to the host to control the working mode of the ultrasound receiving and transmitting circuit.

13. The medical diagnostic ultrasound system according to claim 1, wherein the first transmitting and receiving circuit is configured to communicate with the second transmitting and receiving circuit in a wired manner or a wireless manner of radio frequency.

14. The medical diagnostic ultrasound system according to claim 1, wherein the probe comprises at least one of a freeze key, an auto key, a printing key, and a sensing key, wherein the freeze key is configured to manually control an ON and OFF status of the ultrasound transmitting and receiving circuit, the auto key is configured to cause the medical diagnostic ultrasound system to automatically optimize at least one setting of an ultrasound image, the printing key is configured to directly transmit the ultrasound image to printer to be printed, and the sensing key i configured to manually control whether or not to start the detection device to detect the status of the probe.

15. The medical diagnostic ultrasound system according to claim 1, wherein the medical diagnostic ultrasound system is a portable medical diagnostic ultrasound system.

16. A method for a medical diagnostic ultrasound system to save power, the method comprising:
monitoring whether a probe of the medical diagnostic ultrasound system is being used, and generating a corresponding control signal; and
transmitting the corresponding control signal from a probe side of the medical diagnostic ultrasound system to a host side of the medical diagnostic ultrasound system, to control an ultrasound transmitting and receiving circuit within a host to operate in a freeze mode or scan mode.

17. The method according to claim 16, wherein transmitting the control signal comprises transmitting the control signal from the probe side to the host side in a wired manner or a wireless manner of radio frequency.

18. The method according to claim 16, wherein the medical diagnostic ultrasound system is a portable medical diagnostic ultrasound system.

19. A medical diagnostic ultrasound device comprising:
an ultrasound transmitting and receiving circuit;
a first transmitting and receiving device coupled to the ultrasound transmitting and receiving circuit;
a probe;
a detection device coupled to the probe and configured to detect a usage status of the probe; and
a second transmitting and receiving device coupled to the detection device and configured to transmit a signal representative of the usage status of the probe to the first transmitting and receiving device to control a working mode of the ultrasound transmitting and receiving circuit.

20. The medical diagnostic ultrasound device according to claim 19, further comprising a processor coupled to the detection device and to the second transmitting and receiving device, the processor configured to generate the signal representative of the usage status of the probe.
